(19) United States
(12) Patent Application Publication
(10) Pub. No.: US 2009/0053340 A1
(43) Pub. Date: Feb. 26, 2009

(54) THERAPEUTIC USES OF TOMATO EXTRACTS

(75) Inventors: Lynn Crosbie, Manchester (GB); Niamh O'Kennedy, Manchester (GB)

Correspondence Address: DAVIS WRIGHT TREMAINE LLP/Los Angeles, 865 FIGUEROA STREET, SUITE 2400, LOS ANGELES, CA, 90017-2566, US

(73) Assignee: PROVEXIS NATURAL PRODUCTS LIMITED, Manchester (GB)

(21) Appl. No.: 11/816,122

(22) PCT Filed: Feb. 14, 2006
(86) PCT No.: PCT/GB2006/000521

§ 371 Date: Aug. 13, 2008

(30) Foreign Application Priority Data
Jul. 12, 2007 (GB) 0502985.5

(51) Int. Cl. A61K 36/81

(52) U.S. Cl. 424/777

(57) ABSTRACT

The invention provides the use of a water soluble tomato extract or an active fraction thereof for the manufacture of a medicament for lowering plasma triglyceride levels, the water soluble tomato extract or active fraction thereof being substantially free of lycopene and being substantially free from water-insoluble particulate material.

Also provided by the invention is a method of lowering triglyceride levels in the blood of a patient through the administration of the water soluble tomato extracts.

The present invention relates to the reduction of blood lipid levels, and in particular to the reduction of blood triglyceride levels, by the administration of tomato extracts.

### BACKGROUND TO THE INVENTION

Lipids are water-insoluble bio-molecules that have high solubility in various organic solvents and have a number of biological roles including acting as building blocks for membranes, acting as a fuel source within the body and providing a means of storing energy. There are three major types of membrane lipids, namely phospholipids, glycolipids and cholesterol, of which phospholipids and glycolipids typically contain long chain carboxylic acids commonly referred to as fatty acids. In addition to their role in forming biological membranes, fatty acids also act as a fuel source for cellular activities. Excess fatty acids are stored in the cytoplasm of fat cells in adipose tissue as triacyl esters of glycerol (triglycerides).

Lipids such as cholesterol and triglycerides in general circulate in the blood plasma in the form of lipoproteins. Plasma lipoproteins can be divided into four major classes, based in part on their density which depends on the protein to lipid ratio. The four classes are the chylomicrons, Very Low Density Lipoproteins (VLDL), Low Density Lipoproteins (LDL) and High Density Lipoproteins (HDL).

Chylomicrons are large lipoprotein particles comprising a core of non-polar lipids, (mostly triglycerides) surrounded by a coat of protein, phospholipids and free cholesterol. Chylomicrons have high molecular weights (10⁹ to 10¹⁰) and are typically secreted into the intestinal lymphatic system by the intestinal mucosa following the absorption of a lipid-containing meal. The triglycerides from chylomicrons eventually find their way into storage in adipose tissue.

Very Low Density Lipoproteins (VLDL) contain largely triglycerides, but also some cholesterol and typically have molecular weights of approximately 5×10⁶. VLDLs are secreted by the liver and the triglyceride component of the VLDL is partially derived from dietary carbohydrates. As with chylomicrons, the VLDL triglycerides generally end up stored in adipose tissue.

The fraction of VLDL containing elevated concentrations of cholesterol is often referred to as β VLDL.

Low Density Lipoproteins (LDL) contain the major portion of plasma cholesterol. When LDL is present in increased concentrations, plasma cholesterol concentration is increased while the triglyceride concentration remains relatively normal.

High Density Lipoproteins (HDL) are considerably smaller than the other types of lipoprotein and typically consist largely of proteins and phospholipids. HDL is generally considered to be a beneficial lipoprotein since plasma levels of HDL have been found to be inversely proportional to the risk of atherosclerosis.

The protein components of the lipoproteins are known as apoproteins. In addition to serving as membrane stabilisers, the apoproteins are also required for synthesis and secretion of certain lipoproteins, serve as co-factors in the activation of enzymes that modify the lipoproteins, and interact with specific receptors that remove lipoproteins from the circulation.

Irregular levels of lipids in the blood are associated with a number of disease states and conditions. Dyslipidemia, the generic term used to denote irregular levels of lipids in the blood, can be classified into three commonly encountered types, depending upon the nature of the elevated lipids in the blood plasma. The three general categories are hypercholesterolemia, combined hyperlipidemia and hypertriglyceridemia and these can be further classified phenotypically by electrophoresis into Types I, IIA, IIB, III, IV and V.

Type I hyperlipidemia is characterized by hugely elevated levels of chylomicrons with resultant elevation of triglyceride levels. Type I hyperlipidaemia typically results from either a congenital deficiency of lipoprotein lipase or apo C-II, the apolipoprotein required to activate lipoprotein lipase. The clinical manifestations of this type of hyperlipidemia include eruptive xanthomas and pancreatitis.

Type IIA hyperlipidemia is characterized by elevated levels of LDL cholesterol. Genetic conditions which can cause this are include Familial Hypercholesterolemia, Polygenic Hypercholesterolemia, Familial Combined Hyperlipidemia and Familial Defective Apolipoprotein B-100. Hypercholesterolemia may also be caused by an excess dietary cholesterol intake, or may be a secondary effect of diseases and disorders such as nephritic syndrome, myeloma and hypothyroidism. Individuals suffering from hypercholesterolemia exhibit a high risk of myocardial infarction and are at high risk of developing premature coronary heart disease

Type IIB hyperlipidemia is characterized by elevation of both LDL cholesterol and triglyceride levels. Familial Combined Hyperlipidemia is the most common genetic form of this disorder in which both VLDL and LDL levels are elevated. This disorder affects approximately 1-2% of the American population and studies have shown that approximately 10% of patients with myocardial infarction before the age of 60 come from families with this disease.

Type III hyperlipidemia, also known as Familial Dysbetalipoproteinemia, is characterised by elevated levels of both cholesterol and triglycerides and arises through difficulties in removing triglyceride rich VLDL remnant particles from the blood. The clinical manifestations of this type of hyperlipidemia include the development of tuberous and planar xanthomas. Type III hyperlipidemia is also frequently associated with premature coronary heart disease.

Type IV hyperlipidemia, also known as hypertriglyceridemia, is characterised by elevated levels of triglycerides. Individuals with Type IV hyperlipidemia typically have triglyceride levels of between 250 and 500 mg/dl. Hypertriglyceridemia may be genetic in origin, or may be caused by diseases such as diabetes mellitus or nephrosis. Further causes include the effects of certain medications, and dietary factors such as high sugar and alcohol intake.

Type V hyperlipidemia is characterised by elevated levels of chylomicrons and VLDL, and consequently very high levels of triglycerides. This type of hyperlipidemia, which is due largely to defective lipolysis and an overproduction of VLDL, can be genetic in origin, or can arise as a result of diabetes mellitus, obesity or alcohol consumption. Clinical manifestations include eruptive xanthomas and pancreatitis.

Secondary forms of dyslipidemia are also associated with Diabetes mellitus, hypothyroidism, nephrotic syndrome, obstructive liver disease and the use of certain pharmacologic agents. Agents which can raise LDL or lower HDL levels include progestins, anabolic steroids, corticosteroids and certain antihypertensive agents such as beta-blockers and diuretics. Beta-blockers without intrinsic sympathomimetic activity (ISA) tend to decrease HDL and raise triglyceride levels. Thiazide and loop diuretics can cause a modest and sometimes transient rise in LDL. Birth control pills can cause hypertriglyceridemia in some women.

The role of elevated triglyceride levels in the development of heart disease and mortality associated with the disease has previously been somewhat unclear and, in particular, it has been uncertain whether the increased triglyceride levels are a cause or merely a symptom of the disease. However, over the past few years, evidence has emerged that elevated levels of triglycerides can increase the risk of heart disease developing and can increase mortality in patients with established heart disease.

For example, Jeppesen et al. in Circulation, 1998; 97:1029-1036, described investigations into the effect of triglyceride levels on the risk of ischemic heart disease and disclosed that in middle-aged and older white men, elevated levels of triglycerides may increase the risk of heart attacks occurring. In the Jeppesen study, it was found that men with the highest levels of triglycerides were more than twice as likely to have a heart attack when compared to those with the lowest triglyceride levels. One possible explanation for this is that high levels of triglycerides can influence the size, density distribution and composition of LDL leading to smaller, denser LDL particles, which are more likely to promote the obstructions in the blood vessels that trigger heart attack.

Haim et al., Circulation, 1999, 100:475-482, have reported a connection between elevated triglyceride levels and mortality in patients with established coronary heart disease. In their study, Haim et al. investigated the association between blood lipid levels and mortality in 11,532 patients with heart disease and concluded that, inter alia, elevated triglyceride levels were associated with an increased mortality risk in patients with elevated HDL cholesterol.

The National Heart Lung and Blood Institute (NHLBI), a part of the US National Institute of Health (NIH), has classified blood triglyceride levels as follows:

Triglyceride Blood Levels

| | |
|---|---|
| Normal | Less than 150 mg/dl |
| Borderline high | 150 mg/dl-199 mg/dl |
| High | 200-499 mg/dl |
| Very High | 500 mg/dl or higher |

The US National Cholesterol Education Program (NCEP) in their revised guidelines of 2001 were sufficiently concerned with the health risks posed by elevated levels of triglycerides that they recommend treating even borderline-high triglyceride levels.

It is known that a high consumption of fruits and vegetables is an important preventative measure by which the risk of diseases can be reduced. One factor involved in the initiation and development of diseases is the occurrence of abnormal oxidative processes leading to the generation of hydroxy and peroxy free radicals or compounds. In part, the beneficial effect of eating fruits and vegetables is explained by the antioxidants contained therein that inhibit oxidative reactions. Specific antioxidants known to account for the inhibition include vitamin C, vitamin E and carotenoids including alpha and beta carotenoids, lycopene, lutein, zeanthin, crytoxanthin and xanthophylls.

Considerable effort has been expended in identifying nutritional compounds derived from tomato that have a role in the prevention of disease. Such compounds are disclosed in Abushita et al., Food Chemistry, 1997, 60(2), 207-212 wherein a carotenoid extract of tomato was fractionated and the major components identified as lycopene, beta-carotene and lutein. Studies on tomato have focused on the role of carotenoids, in particular lycopene, in the antioxidant defence against the oxidation of low-density lipoprotein (LDL).

In their International Patent application WO 99/55350, and in EP 1334728, the present applicants disclosed the use of water-soluble extracts of tomato as inhibitors of platelet aggregation.

U.S. Pat. No. 5,502,038 (Medical Research Foundation of Oregon) describes the isolation, synthesis and use of various glycosides containing neotigogenin aglycone moieties that inhibit the absorption of cholesterol and which are useful in the treatment of hypercholesterolemia. Particular compounds of interest are derived from tomato seeds and include neotigogenin trisaccharide. However, the glycosides described in U.S. Pat. No. 5,502,038 would appear to be insoluble or only poorly soluble in water. For example, the compound furostane tetrasaccharide was isolated from tomato seeds by pulverisation of the dried tomato seeds, followed by multiple extractions with methanol. Following chromatography, the furostane tetrasaccharide was converted to neotigogenyl trisaccharide by treatment with β-glucosidase to give a product which is insoluble in water.

Chinese patent application CN 1352941A (Ji Jianjun) discloses a linoleic acid capsule comprising an extract of tomato seed can be used to soften blood vessels and prevent cardiovascular and cerebrovascular diseases and cancer.

Chinese patent application CN 1650951A (Ningbo Jianyong Biolog. Science) discloses that a lycopene mixture prepared from tomato extract and soya oil can be used to prevent arteriosclerosis, myocardial infarction and chronic heart disease.

Friedman et al., Journal of Food Science, Vol. 65, pp 897-900, disclose that feeding red or green tomatoes to hamsters reduces their plasma low-density lipoprotein, cholesterol and triglyceride levels. The article focuses on the ability of various components of red and green tomatoes, such as tomatine (in green tomatoes), lycopene (in red tomatoes), tomato fibre and protein to reduce cholesterol levels but does not contain any information about the effects of the various individual components of the tomato on triglyceride levels. The article concludes that it would be beneficial to make an assessment whether the effects in hamsters are also produced in humans.

### SUMMARY OF THE INVENTION

It has now unexpectedly been found that water-soluble extracts of red tomato that are substantially devoid of lycopene and insoluble fibre reduce plasma levels of triglycerides. The results obtained so far suggest that medicaments containing such extracts may therefore be of use in treating or preventing diseases or conditions arising from or exacerbated by elevated blood levels of triglyceride levels.

Accordingly, in a first aspect, the invention provides the use of a water soluble tomato extract or an active fraction thereof for the manufacture of a medicament for lowering plasma triglyceride levels, the water soluble tomato extract or active fraction thereof being substantially free of lycopene and being substantially free from water-insoluble particulate material.

The tomato extracts of the invention are aqueous extracts from ripe, i.e. red tomatoes, and are water soluble. The term “water soluble” as used herein means that the tomato extracts are soluble at room temperature, e.g. at 25° C. The extracts have also been found to be water soluble at much lower temperatures, for example at temperatures as low as 4° C.

The extracts contain no, or negligible concentrations of, lycopene. For example, the extracts contain less than 0.5% by weight (dry weight) of lycopene, e.g. less than 0.1%, or less than 0.05%, or less than 0.01%, or less than 0.005%, or less than 0.001%, or less than 0.0005%, or less than 0.0001%, by weight (dry weight) of lycopene. The extracts also contain no, or negligible concentrations of, tomatine.

The extracts typically contain no, or negligible concentrations of, tomatine. For example, the extracts contain less than 0.5% by weight (dry weight) of tomatine, e.g. less than 0.1%, or less than 0.05%, or less than 0.01%, or less than 0.005%, or less than 0.001%, or less than 0.0005%, or less than 0.0001%, by weight (dry weight) of tomatine.

The extracts are substantially free from water-insoluble particulate material. Thus, for example, they contain less than 0.5% by weight (dry weight) of water-insoluble particulate material, e.g. less than 0.1%, or less than 0.05%, or less than 0.01%, or less than 0.005%, or less than 0.001%, or less than 0.0005%, or less than 0.0001%, by weight (dry weight) of water-insoluble particulate material. In one embodiment, the extracts contain no water-insoluble particulate material.

The term “active fraction” as used herein refers to a fraction isolated from a tomato extract, which fraction has the ability to reduce blood levels of trigylycerides.

The invention also provides;
A water soluble tomato extract or an active fraction thereof for use in lowering plasma triglyceride levels, the water soluble tomato extract or active fraction thereof being substantially free of lycopene and being substantially free from water-insoluble particulate material.
A composition comprising a water soluble tomato extract or an active fraction thereof for use in lowering plasma triglyceride levels, the water soluble tomato extract or active fraction thereof being substantially free of lycopene and being substantially free from water-insoluble particulate material.
A method of lowering triglyceride levels in the blood of a patient, which method comprises administering to the patient an effective triglyceride lowering amount of a water soluble tomato extract or active fraction thereof, the water soluble tomato extract or active fraction thereof being substantially free of lycopene and being substantially free from water-insoluble particulate material.

An “effective amount” refers to an amount that confers a therapeutic effect on a patient. The therapeutic effect may be objective (i.e. measurable by some test or marker) or subjective (i.e., patient gives an indication of or feels an effect).

Further aspects and embodiments of the invention are as set out below and in the claims appended hereto.

The compositions of the invention may be used for the prophylaxis or treatment of disease states or conditions arising from elevated triglyceride levels. Thus, for example, the compositions can be administered for the purpose of preventing or slowing the onset of coronary heart disease, alleviating coronary heart disease and preventing or reducing mortality in patients suffering from coronary heart disease. The compositions may also be used for the prophylaxis or treatment of hypertriglyceridemia or for the treatment or prophylaxis of conditions such as obesity, eruptive xanthomas and pancreatitis that can arise from elevated triglyceride levels.

Patient populations to whom the compositions may be given include patients suffering from coronary heart disease, diabetes mellitus, hypothyroidism, nephrotic syndrome, obstructive liver disease and obesity.

The patients to whom the extracts, active fractions and medicaments of the invention are administered are typically human patients.

### Preparation and Characterisation of the Extracts

The invention makes use of aqueous extracts of tomatoes.

Such extracts may be prepared by homogenising the flesh of a tomato, with or without its skin, and then filtering the homogenate to remove solids. Substantially all water-insoluble solids are removed, for example by centrifugation and/or filtration.

Alternatively, commercially available tomato pastes may be used as the starting material for the preparation of the extracts. The tomato pastes are typically diluted with water, and then water-insoluble solids are removed, e.g. by centrifugation and/or filtration to give a substantially clear solution.

In each case, removal of the solids has the effect of removing fragments of skin containing lycopene. Thus, the tomato extracts of the invention are water soluble extracts that are substantially free of lycopene.

The aqueous filtrate may be subjected to further fractionation to provide an active fraction containing a compound or compounds responsible for the lipid lowering effect. Alternatively, the filtrate may be evaporated to give a dry water soluble extract.

In one embodiment of the invention, the water soluble extract is in the form of an aqueous solution.

In another embodiment, the water soluble tomato extract is in a dry (e.g. dehydrated) form

Filtration of the tomato homogenate may be accomplished in a single stage, or in a series of filtration steps, starting with a relatively course filtration or centrifugation step to remove larger particles of tomato skin and/or other water-insoluble fragments of tomato flesh. Further filtration steps may then be effected to give a substantially clear solution, e.g. a solution that will pass through a 0.2 p filter without loss of solids.

Thus, in one preferred embodiment of the invention, the tomato extract is a water soluble extract substantially free of lycopene and capable of passing through a 0.2 p filter without loss of solids.

Where the starting material for the preparation of the extracts is a tomato paste, it is preferably one that has been produced by means of a “cold-break”process rather than a “hot-break” process. The terms “cold-break” and “hot-break” are well known in the field of tomato processing and commercially available tomato pastes are typically sold as either hot-break or cold-break pastes. Cold-break pastes are can be prepared by a process involving homogenisation of the tomato followed by a thermal processing step in which the tomatoes are heated to temperature of no more than about 60° C., in contrast to hot-break pastes where the homogenised tomatoes are subjected to thermal processing at temperatures of about 95° C., see for example, Anthon et al., J. Agric. Food Chem. 2002, 50, 6153-6159.

Pharmaceutical and Nutraceutical formulations

The extracts or active fractions thereof may be formulated for oral administration. As such, they can be formulated as solutions, suspensions, syrups, tablets, capsules, lozenges and snack bars, inserts and patches by way of example. Such formulations can be prepared in accordance with methods well known per se.

For example, the extracts or active fractions can be formed into syrups or other solutions for administration orally, for example health drinks, in the presence of one or more excipients selected from sugars, vitamins, flavouring agents, colouring agents, preservatives and thickeners.

Tonicity adjusting agents such as sodium chloride, or sugars, can be added to provide a solution of a particular osmotic strength, for example an isotonic solution. One or more pH-adjusting agents, such as buffering agents can also be used to adjust the pH to a particular value, and preferably maintain it at that value. Examples of buffering agents include sodium citrate/citric acid buffers and phosphate buffers.

Alternatively, the extracts or active fractions thereof can be dried, e.g. by spray drying or freeze drying, and the dried product formulated in a solid or semi solid dosage form, for example as a tablet, lozenge, capsule, powder, granulate or gel.

Instead simple dried extracts can be prepared without any additional components. Alternatively, dried extracts can be prepared by adsorbing on to a solid support; for example a sugar such as sucrose, lactose, glucose, fructose, mannose or a sugar alcohol such as xylitol, sorbitol or mannitol; or a cellulose derivative. Other particularly useful adsorbents include starch-based adsorbents such as cereal flours for example wheat flour and corn flour. For tablet formation, the dried extract is typically mixed with a diluent such as a sugar, e.g. sucrose and lactose, and sugar alcohols such as xylitol, sorbitol and mannitol; or modified cellulose or cellulose derivative such as powdered cellulose or microcrystalline cellulose or carboxymethyl cellulose. The tablets will also typically contain one or more excipients selected from granulating agents, binders, lubricants and disintegrating agents. Examples of disintegrants include starch and starch derivatives, and other swellable polymers, for example crosslinked polymeric disintegrants such as cross-linked carboxymethylcellulose, crosslinked polyvinylpyrrolidone and starch glycolates. Examples of lubricants include stearates such as magnesium stearate and stearic acid. Examples of binders and granulating agents include polyvinylpyrrolidone. Where the diluent is not naturally very sweet, a sweetener can be added, for example ammonium glycyrrhizinate or an artificial sweetener such as aspartame, or sodium saccharinate.

Dried extracts can also be formulated as powders, granules or semisolids for incorporation into capsules. When used in the form of powders, the extracts can be formulated together with any one or more of the excipients defined above in relation to tablets, or can be presented in an undiluted form. For presentation in the form of a semisolid, the dried extracts can be dissolved or suspended in a viscous liquid or semisolid vehicle such as a polyethylene glycol, or a liquid carrier such as a glycol, e.g. propylene glycol, or glycerol or a vegetable or fish oil, for example an oil selected from olive oil, sunflower oil, safflower oil, evening primrose oil, soya oil, cod liver oil, herring oil, etc. Such extracts can be filled into capsules of either the hard gelatine or soft gelatine type or made from hard or soft gelatine equivalents, soft gelatine or gelatine-equivalent capsules being preferred for viscous liquid or semisolid fillings.

Dried extracts can also be provided in a powder form for incorporation in to snack food bars for example fruit bars, nut bars, and cereal bars. For presentation in the form of snack food bars, the dried extracts can be admixed with any one or more ingredients selected from dried fruits such as sun-dried tomatoes, raisins and sultanas, groundnuts or cereals such as oats and wheat.

Dried extracts can be provided in a powder form for reconstitution as a solution. As such they can also contain soluble excipients such as sugars, buffering agents such as citrate and phosphate buffers, and effervescent agents formed from carbonates, e.g. bicarbonates such as sodium or ammonium bicarbonate, and a solid acid, for example citric acid or an acid citrate salt.

In one preferred embodiment, dried extract is provided in powder form optionally together with a preferred solid (e.g. powdered) excipient for incorporation into capsules, for example a hard gelatine capsule.

A solid or semisolid dosage form of the present invention can contain up to about 1000 mg of the dried extract, for example up to about 800 mg.

The extracts can be presented as food supplements or food additives, or can be incorporated into foods, for example functional foods or nutraceuticals.

The compositions of the invention can be presented in the form of unit dosage forms containing a defined concentration of extract or active fraction thereof. Such unit dosage forms can be selected so as to achieve a desired level of biological activity. For example, a unit dosage form can contain an amount of up to 1000 mg (dry weight) of an extract or active fraction, more typically up to 800 mg, for example 50 mg to 800 mg, e.g. 100 mg to 500 mg. Particular amounts of extract or active fraction that may be included in a unit dosage form may be selected from 50 mg, 100 mg, 150 mg, 200 mg, 250 mg, 300 mg, 350 mg, 400 mg, 450 mg, 550 mg, 600 mg, 650 mg, 700 mg, 750 mg and 800 mg.

The compositions of the invention can be included in a container, pack or dispenser together with instructions for administration.

### Pharmaceutical Uses

For the treatment of the diseases and conditions concerned, the quantity of extract or active fraction administered to a patient per day will depend upon the strength of the extract, the particular condition or disease under treatment and its severity, and ultimately it will be at the discretion of the physician. The amount administered however will typically be a non-toxic amount effective to treat the condition in question.

The amount of extract or active fraction administered to a patient typically will vary according to the concentration of the active ingredient or ingredients in the extract. However, a typical daily dosage regime for a human patient suffering from a hyperlipidaemia-mediated disease may be from 0.0001 to 0.1, preferably 0.001 to 0.05 gram per kilogram body weight. When an active fraction is isolated and administered, the amount of solid material administered can be reduced by an amount consistent with the increased purity of the fraction. Typically, administration of at least 100 mg (dry weight or dry weight equivalent) and preferably at least 200 mg, and more usually at least 500 mg of the extract per day to a human patient suffering from elevated triglyceride levels will reduce blood triglyceride levels significantly.

The compositions can be administered in single or multiple dosage units per day, for example from one to four times daily, preferably one or two times daily.

The extracts of the invention can be administered in solid, liquid or semi-solid form. For example, the extracts can be administered in the form of tomato juice or concentrates thereof alone or in admixture with other fruit juices such as orange juice.

The compositions of the invention have triglyceride level reducing activity. As such, the compositions of the invention are useful in the treatment of conditions and disorders in which elevated levels of triglycerides play a part. Such conditions and disorders are described above.

### BRIEF DESCRIPTION OF THE DRAWINGS

The invention will now be illustrated, but not limited, by the following example, and with reference to the accompanying drawings, in which: —

FIG. 1 shows the protocol used in a randomised crossover trial designed by the present inventors to study the effects of tomato extracts on individuals' blood lipid levels.

FIG. 2 compares the effects of consumption of a control substance (2 week period) or tomato extract (4 week period) on plasma lipid levels. The changes from baseline status in total plasma cholesterol, plasma HDL-cholesterol and plasma triglyceride levels are illustrated.

### EXAMPLE 1

### Preparation of a Tomato Extract

A tomato extract for use in the therapeutic method of the invention was prepared using commercially available cold-break tomato paste of 28-30° Brix (i.e. 28-30% solids, w/w) having a browning index (absorbance of a solution of concentration 12.5 g soluble solids/L at 420 nm)<0.350 AU as the starting material. The paste was diluted (1:5) with ultrapure water and large particulate matter was removed by centrifugal filtration followed by clarification using a Westfalia MSB-14 Separator (a centrifugal disk clarifier) at room temperature. Smaller particulate matter was then removed by microfiltration at a temperature not exceeding 45° C., to give a clear straw-coloured solution containing no insoluble spin-down solids and capable of passing through a 0.2μ filter without loss of soluble solids. This solution was concentrated by evaporation to a syrup of 65° Brix, using carefully controlled conditions and a temperature not exceeding 50° C. to limit the progress of non-enzymic browning reactions. A flash pasteurisation step (T=105° C. for 3 seconds) was incorporated at the outset of the evaporation procedure. The final product was characterised by a browning index <0.600 AU, and a microbial total plate count of <1000.

For administration during the human study described below, the concentrated extract was added to an orange juice matrix.

### Summary of Study Protocol

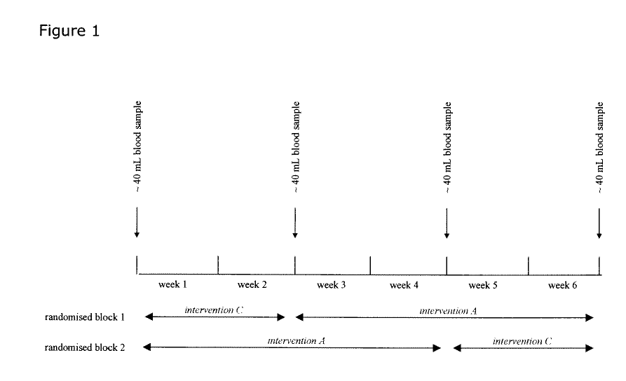

A randomised crossover pilot trial was conducted according to the protocol shown in FIG. 1. The aim of this pilot study was to examine the effects of chronic consumption of an orange juice containing the tomato extract of the present invention on various haematological parameters, compared to a placebo.

One parameter of interest was the composition of blood lipids. A large body of evidence supports a direct relationship between LDL cholesterol and the rate of cardiovascular disease. This includes within-population studies (e.g. Framingham) and between-population studies (i.e. Seven Countries). Familial Hypercholesterolemia, a genetic disorder characterized by high levels of LDL cholesterol, has an exceedingly high rate of premature atherosclerosis. Animals with both spontaneous and diet-induced hypercholesterolemia develop lesions similar to human atherosclerosis.

In this intervention study, levels of different plasma lipid classes—cholesterol, high-density lipoprotein (HDL), low-density lipoprotein (LDL) and triglycerides—were monitored so that any changes in individuals' lipid profiles over the course of the trial could be quantified. The membrane phospholipid composition of some cellular components of the blood was also monitored.

The design of the trial was such that each individual could be placebo-controlled (FIG. 1). After an initial screen to ascertain health status, 22 individuals were asked to attend the Human Nutrition Unit (HNU) of the Rowett Research Institute at Bucksburn, Aberdeen, United Kingdom, once every two weeks over a 6 week period. In order to achieve double-blinding, subjects were randomly assigned to two groups, to undertake interventions 1 and 2 as follows:
Group 1: Intervention 1 (extract enriched functional beverage) for 4 weeks, followed by intervention 2 (placebo) for 2 weeks
Group 2: Intervention 2 (placebo) for 2 weeks followed by intervention 1 (extract enriched functional beverage) for 4 weeks

The enriched functional beverage was prepared by mixing 6 g of the concentrate described above active ingredient and 7.2 g sugar in 200 mL orange juice from concentrate containing flavouring at 0.15%.

A placebo was made by mixing 10.8 g sugar in 200 mL orange juice containing flavouring at 0.15%, without the bioactive ingredient. Both beverages were bottled and pasteurised. One bottle taken around midday comprised the daily dose.

Subjects attended the HNU in the early morning on the first day of their intervention, where they gave a fasted baseline blood sample of approximately 40 mL. This sample was used to obtain a baseline plasma lipid profile for each subject. The subjects were then given the randomly assigned enriched or placebo orange juice to take home and drink at a specified time each day. Two weeks later they returned to the HNU to provide another fasted blood sample, and again once every 2 weeks thereafter, for the duration of the 6 week study. The tomato dose equivalent over the intervention period was 2 tomatoes/day. Measurement of plasma lipids and red cell phospholipid composition were made at each time-point to examine the effect of chronic consumption of the enriched orange juice on these parameters.

Volunteers were requested to abstain from consuming ‘excessive’ amounts of tomatoes, tomato juice or other tomato products (as defined in a ‘diet sheet’) and to keep a daily diary of supplement timing. No additional dietary restrictions were made.

Lipid measurements were carried out using a Kone Autoanalyser on EDTA-anticoagulated plasma. Plasma cholesterol, HDL-cholesterol and triglycerides were quantified. Plasma LDL-cholesterol was calculated by subtraction (LDL-cholesterol=total cholesterol−HDL-cholesterol). Platelet/red blood cell phospholipid composition was measured by GC-MS following extraction by a modified Bligh and Dyer method (results not given).

### Summary of Results

### Plasma Lipid Measurements

Tables 1 and 2 show the quantified lipids (mmol/L) and the % changes from baseline lipid status (A %) at each sampling timepoint for subjects in Group 1 and Group 2, respectively. The different supplementation regimes are shown in these tables as B (baseline i.e. pre-treatment sample), C (control treatment) and E (E1=extract-enriched treatment at 2 weeks, E2=extract-enriched treatment at 4 weeks).

TABLE 1
Group 1

| | Time/ | | | Δ% | | Δ% | | Δ% | | | | Δ% |
|---|---|---|---|---|---|---|---|---|---|---|---|---|
| Subject | wks | Treament | Cholesterol | Chol | HDL- | HDL | Triglyceride | TriG | Chol:HDL | Δ% Chol:HDL | triG:HDL | triG:HDL |
| 2 | 0 | B | 6.7 | 0.0 | 1.36 | 0.0 | 2.02 | 0.0 | 4.9 | 0.0 | 1.5 | 0.0 |
| 2 | 2 | C | 6.42 | −4.2 | 1.52 | 11.8 | 1.87 | −7.4 | 4.2 | −14.3 | 1.2 | −17.2 |
| 2 | 4 | E1 | 6.48 | −3.3 | 1.62 | 19.1 | 1.17 | −42.1 | 4.0 | −18.8 | 0.7 | −51.4 |
| 2 | 6 | E2 | 6.46 | −3.6 | 1.44 | 5.9 | 1.6 | −20.8 | 4.5 | −8.9 | 1.1 | −25.2 |
| 4 | 0 | B | 4.25 | 0.0 | 1.73 | 0.0 | 0.82 | 0.0 | 2.5 | 0.0 | 0.5 | 0.0 |
| 4 | 2 | C | 3.93 | −7.5 | 1.7 | −1.7 | 0.73 | −11.0 | 2.3 | −5.9 | 0.4 | −9.4 |
| 4 | 4 | E1 | 4.06 | −4.5 | 1.64 | −5.2 | 0.8 | −2.4 | 2.5 | 0.8 | 0.5 | 2.9 |
| 4 | 6 | E2 | 4.23 | −0.5 | 1.72 | −0.6 | 0.78 | −4.9 | 2.5 | 0.1 | 0.5 | −4.3 |
| 10 | 0 | B | 7.69 | 0.0 | 2.31 | 0.0 | 1.59 | 0.0 | 3.3 | 0.0 | 0.7 | 0.0 |
| 10 | 2 | C | 7.22 | −6.1 | 2.04 | −11.7 | 1.5 | −5.7 | 3.5 | * | 0.7 | * |
| 10 | 4 | E1 | 7.91 | 2.9 | 2.14 | −7.4 | 1.45 | −8.8 | 3.7 | 11.0 | 0.7 | −1.6 |
| 10 | 6 | E2 | 7.18 | −6.6 | 2.12 | −8.2 | 1.76 | 10.7 | 3.4 | 1.7 | 0.8 | 20.6 |
| 14 | 0 | B | 6.39 | 0.0 | 1.34 | 0.0 | 1.41 | 0.0 | 4.8 | 0.0 | 1.1 | 0.0 |
| 14 | 2 | C | 6.52 | 2.0 | 1.37 | 2.2 | 1.16 | −17.7 | 4.8 | −0.2 | 0.8 | −19.5 |
| 14 | 4 | E1 | 6.74 | 5.5 | 1.57 | 17.2 | 0.96 | −31.9 | 4.3 | −10.0 | 0.6 | −41.9 |
| 14 | 6 | E2 | 6.74 | 5.5 | 1.48 | 10.4 | 1.17 | −17.0 | 4.6 | −4.5 | 0.8 | −24.9 |
| 16 | 0 | B | 5.12 | 0.0 | 1.38 | 0.0 | 0.81 | 0.0 | 3.7 | 0.0 | 0.6 | 0.0 |
| 16 | 2 | C | 5.33 | 4.1 | 1.32 | −4.3 | 0.81 | 0.0 | 4.0 | 8.8 | 0.6 | 4.5 |
| 16 | 4 | E1 | 5.51 | 7.6 | 1.38 | 0.0 | 0.71 | −12.3 | 4.0 | 7.6 | 0.5 | −12.3 |
| 16 | 6 | E2 | 5.59 | 9.2 | 1.39 | 0.7 | 0.98 | 21.0 | 4.0 | 8.4 | 0.7 | 20.1 |

| Subject | Time | Treament | Cholesterol | Δ% | HDL- | Δ% | Triglyceride | Δ% | Chol:HDL | Δ% | triG:HDL | Δ% |
|---|---|---|---|---|---|---|---|---|---|---|---|---|
| 18 | 0 | B | 7.86 | 0.0 | 1.34 | 0.0 | 1.29 | 0.0 | 5.9 | 0.0 | 1.0 | 0.0 |
| 18 | 2 | C | 6.96 | −11.5 | 1.22 | −9.0 | 1.36 | 5.4 | 5.7 | −2.7 | 1.1 | 15.8 |
| 18 | 4 | E1 | 7.29 | −7.3 | 1.3 | −3.0 | 1.14 | −11.6 | 5.6 | −4.4 | 0.9 | −8.9 |
| 18 | 6 | E2 | 7.19 | −8.5 | 1.18 | −11.9 | 1.48 | 14.7 | 6.1 | 3.9 | 1.3 | 30.3 |
| 22 | 0 | B | 6.75 | 0.0 | 1.44 | 0.0 | 1.28 | 0.0 | 4.7 | 0.0 | 0.9 | 0.0 |
| 22 | 2 | C | 6.43 | −4.7 | 1.3 | −9.7 | 1.82 | 42.2 | 4.9 | 5.5 | 1.4 | 57.5 |
| 22 | 4 | E1 | 6.72 | −0.4 | 1.56 | 8.3 | 1.12 | −12.5 | 4.3 | −8.1 | 0.7 | −19.2 |
| 22 | 6 | E2 | 6.71 | −0.6 | 1.55 | 7.6 | 1.18 | −7.8 | 4.3 | −7.6 | 0.8 | −14.4 |

TABLE 2
Group 2

| Subject | Time | Treament | Cholesterol | Δ% | HDL- | Δ% | Triglyceride | Δ% | Chol:HDL | Δ% | triG:HDL | Δ% |
|---|---|---|---|---|---|---|---|---|---|---|---|---|
| 1 | 0 | B | 5.81 | 0.0 | 0.80 | 0.0 | 1.23 | 0.0 | 7.3 | 0.0 | 1.5 | 0.0 |
| 1 | 2 | E1 | 5.26 | −9.5 | 0.78 | −2.5 | 0.98 | −20.3 | 6.7 | −7.1 | 1.3 | −18.3 |
| 1 | 4 | E2 | 5.81 | 0.0 | 0.83 | 3.7 | 1.07 | −13.0 | 7.0 | −3.6 | 1.3 | −16.2 |
| 1 | 6 | C | 5.97 | 2.8 | 0.83 | 3.7 | 1.11 | −9.8 | 7.2 | −1.0 | 1.3 | −13.0 |
| 3 | 0 | B | 5.67 | 0.0 | 1.26 | 0.0 | 1.04 | 0.0 | 4.5 | 0.0 | 0.8 | 0.0 |
| 3 | 2 | E1 | 5.40 | −4.8 | 1.28 | 1.6 | 1.03 | −1.0 | 4.2 | −6.3 | 0.8 | −2.5 |
| 3 | 4 | E2 | 5.44 | −4.1 | 1.34 | 6.3 | 1.39 | 33.7 | 4.1 | −9.8 | 1.0 | 25.7 |
| 3 | 6 | C | 5.28 | −6.9 | 1.38 | 9.5 | 0.93 | −10.6 | 3.8 | −15.0 | 0.7 | −18.4 |
| 5 | 0 | B | 4.58 | 0.0 | 1.06 | 0.0 | 0.92 | 0.0 | 4.3 | 0.0 | 0.9 | 0.0 |
| 5 | 2 | E1 | 4.48 | −2.2 | 1.04 | −1.9 | 0.92 | 0.0 | 4.3 | * | 0.9 | * |
| 5 | 4 | E2 | 4.77 | 4.1 | 1.13 | 6.6 | 0.77 | −16.3 | 4.2 | −2.3 | 0.7 | −21.5 |
| 5 | 6 | C | 4.67 | 2.0 | 1.07 | 0.9 | 0.92 | 0.0 | 4.4 | 1.0 | 0.9 | −0.9 |
| 7 | 0 | B | 6.50 | 0.0 | 1.59 | 0.0 | 1.29 | 0.0 | 4.1 | 0.0 | 0.8 | 0.0 |
| 7 | 2 | E1 | 7.02 | 8.0 | 1.44 | −9.4 | 1.44 | 11.6 | 4.9 | 19.3 | 1.0 | 23.3 |
| 7 | 4 | E2 | 6.73 | 3.5 | 1.57 | −1.3 | 1.06 | −17.8 | 4.3 | 4.9 | 0.7 | −16.8 |
| 7 | 6 | C | 6.53 | 0.5 | 1.38 | −13.2 | 1.38 | 7.0 | 4.7 | 15.7 | 1.0 | 23.3 |
| 9 | 0 | B | 5.89 | 0.0 | 1.23 | 0.0 | 1.92 | 0.0 | 4.8 | 0.0 | 1.6 | 0.0 |
| 9 | 2 | E1 | 6.31 | 7.1 | 1.10 | −10.6 | 1.59 | −17.2 | 5.7 | * | 1.4 | * |
| 9 | 4 | E2 | 6.25 | 6.1 | 1.08 | −12.2 | 1.94 | 1.0 | 5.8 | * | 1.8 | * |
| 9 | 6 | C | 5.94 | 0.8 | 1.17 | −4.9 | 1.74 | −9.4 | 5.1 | * | 1.5 | * |
| 11 | 0 | B | 5.31 | 0.0 | 1.85 | 0.0 | 0.63 | 0.0 | 2.9 | 0.0 | 0.3 | 0.0 |
| 11 | 2 | E1 | 5.77 | 8.7 | 1.81 | −2.2 | 0.61 | −3.2 | 3.2 | 11.1 | 0.3 | −1.0 |
| 11 | 4 | E2 | 5.63 | 6.0 | 1.85 | 0.0 | 0.57 | −9.5 | 3.0 | 6.0 | 0.3 | −9.5 |
| 11 | 6 | C | 5.27 | −0.8 | 1.75 | −5.4 | 0.59 | −6.3 | 3.0 | 4.9 | 0.3 | −1.0 |
| 17 | 0 | B | 6.40 | 0.0 | 1.16 | 0.0 | 1.78 | 0.0 | 5.5 | 0.0 | 1.5 | 0.0 |
| 17 | 2 | E1 | 7.40 | 15.6 | 1.23 | 6.0 | 1.55 | −12.9 | 6.0 | 9.0 | 1.3 | −17.9 |
| 17 | 4 | E2 | 6.82 | 6.6 | 1.33 | 14.7 | 0.96 | −46.1 | 5.1 | −7.1 | 0.7 | −53.0 |
| 17 | 6 | C | 6.76 | 5.6 | 1.26 | 8.6 | 1.51 | −15.2 | 5.4 | −2.8 | 1.2 | −21.9 |
| 19 | 0 | B | 5.03 | 0.0 | 0.95 | 0.0 | 1.46 | 0.0 | 5.3 | 0.0 | 1.5 | 0.0 |
| 19 | 2 | E1 | 5.10 | 1.4 | 1.10 | 15.8 | 1.60 | 9.6 | 4.6 | −12.4 | 1.5 | −5.4 |
| 19 | 4 | E2 | 5.04 | 0.2 | 1.18 | 24.2 | 1.22 | −16.4 | 4.3 | −19.3 | 1.0 | −32.7 |
| 19 | 6 | C | 4.58 | −8.9 | 1.15 | 21.1 | 1.85 | 26.7 | 4.0 | −24.8 | 1.6 | 4.7 |
| 21 | 0 | B | 6.09 | 0.0 | 0.91 | 0.0 | 1.66 | 0.0 | 6.7 | 0.0 | 1.8 | 0.0 |
| 21 | 2 | E1 | 5.97 | −2.0 | 0.96 | 5.5 | 1.10 | −33.7 | 6.2 | −7.1 | 1.1 | −37.2 |
| 21 | 4 | E2 | 6.24 | 2.5 | 0.99 | 8.8 | 1.43 | −13.9 | 6.3 | −5.8 | 1.4 | −20.8 |
| 21 | 6 | C | 6.73 | 10.5 | 0.99 | 8.8 | 1.84 | 10.8 | 6.8 | 1.6 | 1.9 | 1.9 |

16 full data sets were obtained from the 22 subjects recruited onto the trial. Table 3 summarises the baseline plasma lipid status of the randomised subject Groups 1 and 2. It can be seen that Group 1 and Group 2 had similar plasma lipid profiles at baseline. Overall, the plasma lipid levels were higher than current Department of Health recommendations, with total cholesterol (Chol) greater than 5 mmol/L. In addition, the cholesterol:HDL-cholesterol ratio (Chol:HDL) was greater than 4 for both groups.

TABLE 3
Baseline plasma lipid profiles for Group 1 and Group 2. Mean
values are expressed in mmol/L, and are given with their standard
errors.

| | Choles- | HDL- | | Chol:HDL | HDL:TriG |
|---|---|---|---|---|---|
| | terol | Cholesterol | Triglycerides | ratio | ratio |
| Group 1 | 6.39 ± 0.5 | 1.56 ± 0.1 | 1.32 ± 0.2 | 4.2 ± 0.4 | 0.9 ± 0.1 |
| n = 7 | | | | | |
| Group 2 | 5.70 ± 0.2 | 1.20 ± 0.1 | 1.33 ± 0.1 | 5.0 ± 0.4 | 1.2 ± 0.2 |
| n = 9 | | | | | |

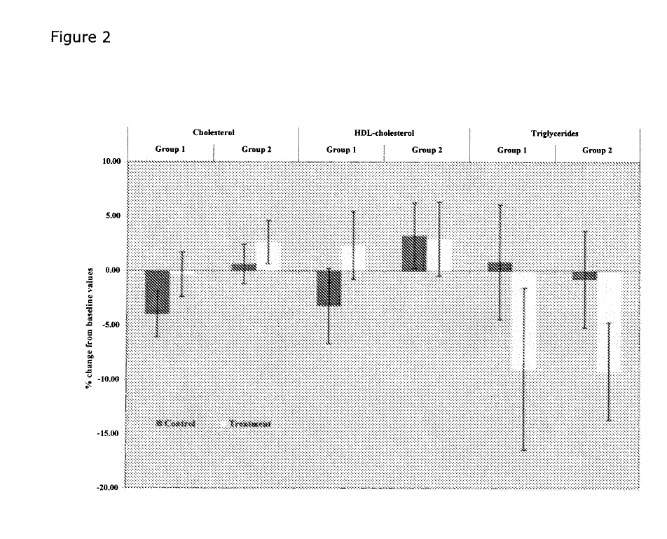

The summary table below (Table 4) shows changes observed from the Group 1 and Group 2 baseline values for total plasma cholesterol, HDL-cholesterol and triglycerides, for the treatment (E) and control (C) supplementation periods. This is illustrated in the graph in FIG. 2. No difference was seen between extract and control treatments for total cholesterol or HDL-cholesterol. However plasma triglycerides were decreased in both groups after extract treatment, compared to control (see Table 4 and FIG. 2).

TABLE 4
Changes from baseline status after supplementation with
control or extract-enriched orange juice. Mean values are expressed as
percentage changes from baseline, and are given with their standard
errors.

| | | Group 1 | Group 2 |
|---|---|---|---|
| | | n = 7 | n = 9 |
| | | % change ± SEM | % change ± SEM |
| Cholesterol | control | −3.98 ± 2.0 | 0.62 ± 2.0 |
| | extract | −0.33 ± 2.1 | 2.63 ± 1.8 |
| HDL- | control | −3.21 ± 3.1 | 3.24 ± 3.4 |
| cholesterol | extract | 2.36 ± 3.4 | 2.96 ± 3.0 |
| Triglycerides | control | 0.83 ± 7.4 | −0.74 ± 4.5 |
| | extract | −8.99 ± 5.3 | −9.19 ± 4.4 |

The data demonstrate that chronic consumption of an orange juice enriched with a tomato extract can cause a reduction in plasma triglyceride levels. It was observed that subjects showing the largest individual responses to the enriched orange juice were among those with the highest baseline triglyceride:HDL-cholesterol ratio (see Tables 1, 2 and 3).

Triglycerides are fats that come from the diet or are manufactured by the body, and account for approximately 95% of the body's fatty tissue. The major triglyceride-containing lipoproteins are called very low density lipoproteins (VLDL). High plasma concentrations of triglycerides (or VLDL) are associated with increased risk of heart disease. The ratio of triglycerides to HDL-cholesterol can be a strong predictor of heart attack in some populations (men, diabetics, hypertensives) suggesting a complex metabolic interaction between triglycerides and other blood lipids. High triglycerides may reduce activity of fat-degrading enzymes, resulting in high levels of VLDL and low levels of HDL.

The variance observed between measurements was high, reflecting both the different baseline lipid status of the subjects and the free diet (absence of dietary control) of the subjects during the study period. For this reason the onset and persistence of the observed effect on triglycerides cannot yet be concluded, as individuals showed different patterns of response. Some (e.g. subject 14) showed a strong effect at t=2 weeks which appeared to be reduced at t=4 weeks, while others (e.g. subject 17) displayed an opposite pattern of behaviour. Dietary control will be necessary to elucidate the observed effects further.

This pilot trial has shown that consumption of the extract-enriched orange juice over a 4 week period did cause some changes in subjects' plasma lipid levels, in particular by reducing fasted plasma triglyceride levels. Therefore long-term consumption of the tomato extract could result in beneficial changes in plasma lipid profiles.

### EXAMPLE 2

### Formulations

### Capsule Formulation

A capsule formulation is prepared by freeze drying a tomato extract as described in Example 1 and filling the resulting freeze dried powder into a hard gelatin capsule shell to give a capsule content of 800 mg per capsule.

### Capsules Containing Diluted Tomato Extract

To the aqueous tomato extract of Example 1 is added a diluent selected from sucrose, lactose and sorbitol. The resulting mixture is then freeze dried to give a powder which is filled into hard gelatin capsule shells to give a capsule content of 800 mg per capsule (200 mg tomato extract and 600 mg diluent).

### EQUIVALENTS

The foregoing examples are presented for the purpose of illustrating the invention and should not be construed as imposing any limitation on the scope of the invention. It will readily be apparent that numerous modification and alterations may be made to the specific embodiments of the invention described above and illustrated in the examples without departing from the principles underlying the invention. All such modifications are intended to be embraced by this application.

1. (canceled)

2. A water soluble tomato extract or an active fraction thereof for use in lowering plasma triglyceride levels, the water soluble tomato extract or active fraction thereof being substantially free of lycopene and being substantially free from water-insoluble particulate material.

3. A composition comprising a water soluble tomato extract or an active fraction thereof for use in lowering plasma triglyceride levels, the water soluble tomato extract or active fraction thereof being substantially free of lycopene and being substantially free from water-insoluble particulate material.

4. A method of lowering triglyceride levels in the blood of a patient, which method comprises administering to the patient an effective triglyceride lowering amount of a water soluble tomato extract or active fraction thereof, the water soluble tomato extract or active fraction thereof being substantially free of lycopene and being substantially free from water-insoluble particulate material.

5. The water soluble tomato extract of claim 2 wherein the water soluble tomato extract is substantially free from particulate material.

6. The water soluble tomato extract of claim 2 wherein an aqueous solution of the water soluble tomato extract is capable of passing through a 0.2μ filter without loss of solids.

7. The water soluble tomato extract of claim 2 wherein the water soluble tomato extract has been dehydrated to give a water soluble dried extract.

8. The water soluble tomato extract of claim 2 wherein the water soluble tomato extract has been prepared from whole tomato or from a cold-break tomato paste.

9. The water soluble tomato extract of claim 2 wherein the lowering of plasma triglyceride levels is effected for the purpose of preventing or slowing the onset of coronary heart disease and/or alleviating coronary heart disease and preventing or reducing mortality in patients suffering from coronary heart disease, or the purpose of the prophylaxis or treatment of hypertriglyceridemia or the treatment or prophylaxis of conditions such as obesity, eruptive xanthomas and pancreatitis that can arise from elevated triglyceride levels.

10. The water soluble tomato extract of claim 2 wherein the water soluble tomato extract is in the form of an aqueous solution.

11. The water soluble tomato extract of claim 2 wherein the water soluble tomato extract is in a dry form.

12. The composition of claim 3 wherein the water soluble tomato extract is substantially free from particulate material.

13. The composition of claim 3 wherein an aqueous solution of the water soluble tomato extract is capable of passing through a 0.2μ filter without loss of solids.

14. The composition of claim 3 wherein the water soluble tomato extract has been dehydrated to give a water soluble dried extract.

15. The composition of claim 3 wherein the water soluble tomato extract has been prepared from whole tomato or from a cold-break tomato paste.

16. The composition of claim 3 wherein the lowering of plasma triglyceride levels is effected for the purpose of preventing or slowing the onset of coronary heart disease and/or alleviating coronary heart disease and preventing or reducing mortality in patients suffering from coronary heart disease, or the purpose of the prophylaxis or treatment of hypertriglyceridemia or the treatment or prophylaxis of conditions such as obesity, eruptive xanthomas and pancreatitis that can arise from elevated triglyceride levels.

17. The composition of claim 3 wherein the water soluble tomato extract is in the form of an aqueous solution.

18. The composition of claim 3 wherein the water soluble tomato extract is in a dry form.

19. The method of claim 4 wherein the water soluble tomato extract is substantially free from particulate material.

20. The method of claim 4 wherein an aqueous solution of the water soluble tomato extract is capable of passing through a 0.2μ filter without loss of solids.

21. The method of claim 4 wherein the water soluble tomato extract has been dehydrated to give a water soluble dried extract.

22. The method of claim 4 wherein the water soluble tomato extract has been prepared from whole tomato or from a cold-break tomato paste.

23. The method of claim 4 wherein the lowering of plasma triglyceride levels is effected for the purpose of preventing or slowing the onset of coronary heart disease and/or alleviating coronary heart disease and preventing or reducing mortality in patients suffering from coronary heart disease, or the purpose of the prophylaxis or treatment of hypertriglyceridemia or the treatment or prophylaxis of conditions such as obesity, eruptive xanthomas and pancreatitis that can arise from elevated triglyceride levels.

24. The method of claim 4 wherein the water soluble tomato extract is in the form of an aqueous solution.

25. The method of claim 4 wherein the water soluble tomato extract is in a dry form.
